SEQUENCE LISTING (2 sequences)

SEQ ID NO: 1 - DNA, 1090 bp, Trichoderma reesei
aaagcggcgaatgactcttcggggttggccagaaacaacgacgaccactgagggatcgggacgatgtacatctactgcgatatgccgggctcgaatgtgactgcacacaccacacacacacacacacacggacacatctgagcagtactatcaaagcggtgggagtgcatctgcactgggcacccttactgtaggtaggaaggatacctaaggtagagggctcgctgctgcaagttgatatgacggccaccaatcggaagggtgtcgtatctcacatcaccagcgtcaggacacgcttcacaaaattcccacaagttccagttgaaatgacccgtaggagagcgcctcgagggacagaatgtacagtactatactacctaccttagatgccatcaccccttcgcaggccatggaatttctggcaagtcgcacccttcagccatgcctgtccgccatcatcataccagatcgtgtcgtggcgggcacttgggaaggacgagctccaaccgccgccgctgattggcctgatccgccacagccactggcgctcgcggcgaggaaaaaaaaaaatcttgcgggtgtgcagcagcattgagtggaaagcgggagccgctgcacgacgctgattcgtccgggctgcctcgttagggctagaccagcaggcgggcgccgcttattttgcccctcctcaggctttttttcacaagccccgttttgctgccccgcgcgtggcagaaaaaaaaaatacagcacgagccactggcacttgcacccctccacccctccgccgaccaccttttaaatctcctcctcccaccatctgctttcgaaattttctctcttctgctcttcgtctcgcatacccggttcaagcatccgatctgcgaatttgtatgtgctgcctctccctctgaccttctggtctggtgataccatcctccctcagtttggatcatcgccttattcttcttccctcttctgcatctgcttcctgctcgtttgaggaacatcgccagctgactctgcttgcctcgcagcgatctagtcaagaacaacacagctctcacgctacatcacacaaaccgtcaaa

SEQ ID NO: 2 - protein, 790 aa, Trichoderma reesei
MAPQDYDDDDFVDQYDEDLGEEEDELSPEDKIAMQKGTEEVRKALGEEASKVTVAQIQEALWHYYYDIDKSVAYLTRTFIAPPPKPAPKAAQKPAPKKAPEGKNGALSFFASGASFKPRIPPNFFDDMPWLNIPRDRETTFIEPPRLCGGLLGGTGSAPKVSKLQALAAMRKRKIEEKKAQENPDSLEGEVTKLALSDANQENTGTKSKIQRRNSSTDSSPPQEQPQKQGQNIPTASTDNSLGERVTLPTATESKTDHIIPQAPKPVMSRSHAPSAFAQALFESDSEADQRHRRDVFAMPYTQSSLFVASVFSEPSPDDIVLAARAKATPKTPKKSQKNEETSKDGALAGGVANLKVTDSPPPKSKGIDVIKEFEQSASKKHASFVVVGHVDAGKSTLMGRLLLELKLVQERTVDKYRRQAEKMGKTSFALAWVFDQRSEERDRGVTIDIATNHFETDSTNFTILDAPGHRDFVPNMIAGASQADFAVLVIDASTGAYEKGLKGQTKEHVLLLRSLGVQRIVVAVNKLEAVGWSQERFQEISEEISGFLTGLGFQEKSIKFIPISGLNGDNIVKRSEDEACSWYTGPTLIEGLEASVPSTVRSLQKPFRMAISEIFRSQQGTTTLAGRIDSGTIQVGDALLVQPSGESAHVRSIMLDTETRDWAVAGQNVSIGLAGIDPVHVKVGDIVCHTKDPINVGDTFTMKAMAFAHLMPMPVDLHRGRLHAAGQIQSIAALLDKATGEVIKKKPKVVQPGSVARVLIKLDTKVPLEAGQRVVIRTGGETIAAGLLE

(19) United States
(12) Patent Application Publication
(10) Pub. No.: US 2024/0384290 A1
(43) Pub. Date: Nov. 21, 2024

(54) Insertion of Multicopies of a Gene of Interest into the Genome of a Fungus

(71) Applicant: IFP ENERGIES NOUVELLES, RUEIL MALMAISON (FR)

(72) Inventors: Hugues Mathis, Rueil Malmaison (FR); Frédérique Bidard-Michelot, Rueil Malmaison (FR)

(73) Assignee: IFP ENERGIES NOUVELLES, Rueil Malmaison (FR)

(21) Appl. No.: 18/288,280

(22) PCT Filed: Apr. 29, 2022
(86) PCT No.: PCT/FR2022/050826

§ 371 Date: Oct. 25, 2023

(30) Foreign Application Priority Data
Apr. 30, 2021 (FR) 2104588

(51) Int. Cl. C12N 15/85; C12N 1/14

(52) U.S. Cl. CPC C12N 15/85; C12N 1/14; C12N 2800/10

(57) ABSTRACT

The invention relates to a method for inserting several copies of a gene of interest into the genome of a fungus, said method comprising a step of transforming said fungus by using a vector comprising an expression cassette, said cassette comprising: (1) a TEF1 promoter identical to the endogenous TEF1 promoter of the species of fungus, (2) a gene of interest, and (3) a terminator that is not the endogenous TEF1 terminator of the fungus species. The invention also relates to the fungi obtained by such a method, and to the various uses of the strains modified in this manner.

(no figure).

This invention relates to a method for inserting several copies of a gene of interest into the genome of a fungus. This invention also relates to the fungus strains thus obtained, and the various uses for such a modified fungus strain.

### CONTEXT OF THE INVENTION

Fungus strains are used for the production of proteins of interest. For example, fungus strains belonging to the Trichoderma genus, in particular Trichoderma reesei, are now predominantly used for enzyme production. These enzymes, for example cellulases, are in fact used to hydrolyze cellulosic or lignocellulosic biomass into simple sugars. The enzymes produced by filamentous fungi are therefore useful in production chains for second-generation biofuels or bio-based products derived from sugars originating from (ligno)cellulosic biomass.

In order to improve the production of second-generation biofuels or bio-based products, consideration has been given to improving the production of cellulases.

For example, patent EP 448 430 B1 describes an optimized industrial production of cellulases by Trichoderma reesei. This production is carried out in a fed-batch protocol using a feed solution containing lactose as the sugar that induces protein production. This fermentation process comprises two stages: a first stage of fungus growth in the presence of excess carbon source, and a second stage of enzyme production by means of adding an inducer into the medium with an optimized flow rate (fed-batch mode). These steps are carried out in a liquid medium in bioreactors while stirring and in the presence of oxygen because the fungus is strictly aerobic. Another example of an optimized process for producing cellulases is described in patent EP 2 744 899 B1.

In addition to optimization of cellulase production processes, consideration has also been given to genetically modifying fungal strains in order to alter their production capacity. Thus, international patent application PCT/FR2016/053601 describes the overexpression of the TrAZF1 gene in a strain in order to improve its ability to produce a protein of interest, in particular a cellulolytic enzyme.

Fermentation processes and strains useful for improving the production of cellulolytic enzymes by filamentous fungi, and doing so from bio-based products but also second generation biofuels, are therefore already described in the prior art.

Fungi can also be used to produce proteins that are useful in other types of industries, such as the food, cosmetics, or pharmaceutical industry (for example, see Rantasalo, A., Vitikainen, M., Paasikallio, T. et al. Novel genetic tools that enable highly pure protein production in Trichoderma reesei. Sci Rep 9, 5032 (2019); or patent EP3405580).

The TEF1 promoter is known to be a strong promoter in fungi (Gao S, Zhou H, Zhou J, Chen J. Promoter-Library-Based Pathway Optimization for Efficient (2S)-Naringenin Production from p-Coumaric Acid in Saccharomyces cerevisiae. J Agric Food Chem. 2020 Jun. 24;68(25):6884-6891. doi:10.1021/acs.jafc.0c01130.Epub 2020 Jun. 11).

The use of this TEF1 promoter has also been described or suggested for promoting the expression of genes of interest (see for example US 20080044878, U.S. Pat. Nos. 9,732,338, 6,011,147, WO 2018067068, U.S. Pat. No. 8,071,298, or Kitamoto, N., Matsui, J., Kawai, Y. et al. Utilization of the TEF1-a gene (TEF1) promoter for expression of polygalacturonase genes, pgaA and pgaB, in Aspergillus oryzae. Appl Microbiol Biotechnol 50, 85-92 (1998); Zhang J, Cai Y, Du G, Chen J, Wang M, Kang Z. Evaluation and application of constitutive promoters for cutinase production by Saccharomyces cerevisiae. J Microbiol 2017 Jul; 55(7):538-544 Epub 2017 Jun. 30; Umemura M, Kuriiwa K, Dao LV, Okuda T, Terai G. Promoter tools for further development of Aspergillus oryzae as a platform for fungal secondary metabolite production. Fungal Biol Biotechnol. 2020 Mar 23; 7:3; Fang F, Salmon K, Shen MW, Aeling KA, Ito E, Irwin B, Tran UP, Hatfield GW, Da Silva NA, Sandmeyer S. A vector set for systematic metabolic engineering in Saccharomyces cerevisiae. Yeast. 2011 February; 28(2):123-36. Epub 2010 Oct 8; Tiina Nakari, Edward Alatalo and Merja E. Penttila, Isolation of Trichoderma reesei genes highly expressed on glucose containing media: characterization of the TEF1 gene encoding translation elongation factor 1 a, Gene, 136(1993)313-318, 1993 Elsevier Science Publishers B.V; Partow S, Siewers V, Bjørn S, Nielsen J, Maury J. Characterization of different promoters for designing a new expression vector in Saccharomyces cerevisiae. Yeast. 2010 Nov; 27(11):955-64).

Also, the multicopy integration of expression vectors into the genome of a fungus has already been described; for example, see EP0778348 or WO1991002803.

Genetic engineering techniques, involving in particular the TEF1 promoter and the expression of genes of interest, have therefore already been described in the prior art.

Furthermore, to target an insertion at a well-defined locus, it is usually necessary for the transgene to be flanked by sequences upstream and downstream of the target locus. The size (number of base pairs) of these flanking regions depends on the organisms. For those with the ability to carry out homologous recombination, these flanking sequences can be short: for example, in the case of Saccharomyces cerevisiae, around twenty base pairs are sufficient to target a locus (Janke C, Magiera MM, Rathfelder N, Taxis C, Reber S, Maekawa H, Moreno-Borchart A, Doenges G, Schwob E, Schiebel E, Knop M. A versatile toolbox for PCR-based tagging of yeast genes: new fluorescent proteins, more markers and promoter substitution cassettes. 2004 August; 21(11):947-62). Conversely, in other fungi such as Trichoderma reesei, these flanking sequences must be 1000 base pairs and even under these conditions, the probability that insertion takes place at the desired site is low.

Gene editing techniques can reduce the size of the flanking regions to 200 base pairs and increase the probability of insertion at the site.

Nevertheless, there is still a need for new methods enabling the improved expression of genes of interest and/or methods for producing proteins of interest, which are as efficient as possible, in particular in Trichoderma reesei.

The present invention is based here on results of the Inventors showing that the TEF1 promoter can be used for multicopy insertion of a gene of interest. Each functional copy inserted leads to an increase in the expression of said gene of interest, thus increasing the production capacity of the protein of interest. The results of the present invention also show that it is possible to carry out insertion successfully, in particular in Trichoderma reesei, at the target locus (region 5′ of the TEF1 promoter) by using only one flanking region of the insertion site in the expression cassette according to the invention (instead of the usual two).

### BRIEF DESCRIPTION OF THE INVENTION

The present invention thus relates to a method for inserting several copies of a gene of interest into the genome of a fungus, said method comprising a step of transforming said fungus by using a vector comprising an expression cassette, said cassette comprising:
a TEF1 promoter identical to the endogenous TEF1 promoter of the fungus species,
a gene of interest,
a terminator that is not the endogenous TEF1 terminator of the fungus species.

The invention also relates to the use of an expression cassette for inserting several copies of a gene of interest into the genome of a fungus, said expression cassette comprising:
a TEF1 promoter identical to the endogenous TEF1 promoter of the fungus species,
a gene of interest,
a terminator that is not the endogenous TEF1 terminator of the fungus species.

The invention also relates to a method for producing a protein of interest, comprising the following steps:
(i) a step of transforming a fungus by using a vector comprising an expression cassette comprising: (1) a TEF1 promoter identical to the endogenous TEF1 promoter of the fungus species, (2) a gene of interest encoding said protein of interest, and (3) a terminator that is not the endogenous TEF1 terminator of the fungus species,
(ii) a step of culturing the fungus transformed in step (i), under conditions enabling production of the protein of interest.

The invention also relates to a fungus strain comprising, in its genome, several copies of a gene of interest under the control of a TEF1 promoter identical to the endogenous TEF1 promoter of the genetically modified fungus species.

### DESCRIPTION OF THE INVENTION

In a first aspect, the invention relates to a method for inserting several copies of a gene of interest into the genome of a fungus, said method comprising a step of transforming said fungus by using a vector comprising an expression cassette, said cassette comprising:
a TEF1 promoter identical to the endogenous TEF1 promoter of the fungus species,
a gene of interest,
a terminator that is not the endogenous TEF1 terminator of the fungus species.

In the invention, the term “TEF1” refers to Translation Elongation Factor 1. It is a GTPase-like protein that is involved in protein biosynthesis. In Trichoderma reesei, the TEF1 protein, encoded by the TEF1 gene, is represented by SEQ ID NO: 2. Expression of the TEF1 gene is essentially constitutive (Tiina Nakari, Edward Alatalo and Merja E. Penttila, Isolation of Trichoderma reesei genes highly expressed on glucose containing media: characterization of the TEF1 gene encoding translation elongation factor 1a, Gene, 136(1993)313-318, 1993 Elsevier Science Publishers B.V). More particularly, in the invention the term “TEF1” refers to “TEF1 alpha”.

In the invention, TEF1 promoter means the promoter under which the TEF1 gene is naturally controlled, in a non-genetically modified organism. Typically, in Trichoderma reesei, the TEF1 gene which encodes the TEF1 protein of SEQ ID NO: 2 is under the control of the TEF1 promoter represented by the sequence SEQ ID NO: 1. According to one embodiment, in the method according to invention, the TEF1 promoter is represented by SEQ ID NO: 1 or a sequence having a percentage identity of at least 60% with SEQ ID NO: 1, preferably at least 80%. According to the invention, the expression “percentage identity of at least 60% with SEQ ID NO: 1” means all the values between 60% and 100%, in particular the values of 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% and 100%, preferably at least 80%, at least 85%, at least 90%, at least 95%, even more particularly at least 98%, at least 99%. A person skilled in the art knows how to calculate a percentage identity between two sequences. For example, according to the invention, the percentage identity of a given sequence relative to SEQ ID NO: 1 means the percentage identity over the total length of the sequences. The percentage thus corresponds to the number of identical nucleotides (residues where appropriate) between this given sequence and SEQ ID NO: 1, divided by the number of nucleotides (residues where appropriate) in the longer of the two sequences. According to one embodiment, in the method according to the invention, the TEF1 promoter can also be understood as being a sequence having a percentage identity of less than 60% with SEQ ID NO: 1 while retaining an identical function. This includes promoters orthologous to the TEF1 promoter in fungi other than Trichoderma, in particular Trichoderma reesei, which have sequences quite different from SEQ ID NO: 1, although performing the same function as the TEF1 promoter in Trichoderma reesei (namely the TEF1 promoter of the TEF1 gene encoding a translation elongation factor 1, more particularly the translation elongation factor 1 alpha). This can also be the case if the promoter is mutated (on nucleotides not essential to its promoter function). According to one embodiment, the TEF1 promoter can also mean the promoter controlling a gene encoding an orthologous or variant protein of the TEF1 protein, represented by SEQ ID NO: 2. Thus, the TEF1 promoter can also mean the promoter of a gene encoding a protein of SEQ ID NO: 2 or a protein having a percentage identity of at least 80% with SEQ ID NO: 2, preferably at least 85%, at least 90%, at least 95%, at least 98%, at least 99%. The expression “at least 80%” represents all values between 80% and 100%, namely 80%, 81%, 82%, 83%, 84%, 85%, 86%, 87%, 88%, 89%, 90%, 91%, 92%, 93%, 94%, 95%, 96%, 97%, 98%, 99% and 100%.

According to the invention, a promoter represented by a sequence having a percentage identity of at least 60% with SEQ ID NO: 1, preferably represents a variant promoter or an orthologous promoter in a fungus other than Trichoderma reesei.

In the expression cassette according to the invention, a TEF1 promoter identical to the endogenous TEF1 promoter of the fungus species is used. The term “endogenous TEF1 promoter of the fungus species” also refers to the “native TEF1 promoter” of the fungus species. Typically, the TEF1 promoter in Trichoderma reesei being represented by SEQ ID NO: 1, then it is a promoter of SEQ ID NO: 1 which must be used in the expression cassette intended to be integrated when the host fungus is Trichoderma reesei. In other words, the TEF1 promoter which must be used, in the expression cassette according to the invention, must be identical to the TEF1 promoter naturally present in the fungus species which is to be transformed. For example, if the host fungus used is Saccharomyces cerevisiae, then the TEF1 promoter to be used in the expression cassette is the endogenous/native TEF1 promoter of Saccharomyces cerevisiae. The term “TEF1 promoter identical to the endogenous TEF1 promoter” means that the sequence of the TEF1 promoter used is strictly identical to that of the endogenous promoter, but also that the sequence of the TEF1 promoter used may vary at certain nucleotides (not essential to the function of the promoter).

The TEF1 promoter present in a fungus species can easily be determined, for example using databases such as FungiDB, by sequencing the fungus genome or by any other appropriate technique. According to one embodiment, the method according to the invention may thus comprise, before the transformation step, an optional step of determining the sequence of the TEF1 promoter naturally present in the fungus that is to be transformed.

According to the invention, the term “multiple copies” means at least two copies. According to a preferred embodiment, between 2 and 15 copies of the gene of interest are inserted into the genome of the transformed fungus, preferably between 2 and 11 copies. Between 2 and 15 copies of course means all values comprised between 2 and 15, i.e. 2, 3, 4, 5, 6, 7, 8, 9, 10, 11, 12, 13, 14 and 15.

The transformation step refers to any technique enabling integration of the expression cassette into the genome of the fungus. Such a technique is well known to those skilled in the art and includes, for example, biolistics (Te'o VS, Bergquist PL, Nevalainen KM., (2002). Biolistic transformation of Trichoderma reesei using the Bio-Rad seven barrels Hepta Adapter system. J Microbiol Methods. 51(3):393-9) or the protoplast method (Penttilä M, Nevalainen H, Rättö M, Salminen E, Knowles J., (1987). A versatile transformation system for the cellulolytic filamentous fungus Trichoderma reesei. Gene. 61(2):155-64).

According to the invention, a “vector” means any DNA sequence into which it is possible to insert fragments of foreign nucleic acid, the vectors making it possible to introduce foreign DNA into a host cell (here the fungus). Examples of vectors are plasmids, cosmids, yeast artificial chromosomes (YAC), bacterial artificial chromosomes (BAC) and bacteriophage P1-derived artificial chromosomes (PAC), virus-derived vectors. The vector according to the invention is an integrative vector (preferably an integrative plasmid), enabling integration of the expression cassette into the genome of the fungus. According to one particular embodiment, the vector does not comprise a fungal origin of replication.

According to the invention, an “expression cassette” comprises a DNA fragment (the gene of interest) placed under the control of the elements necessary for its expression. Said DNA fragment is thus placed under the control of the TEF1 promoter. According to the invention, the expression cassette comprises at least: (1) a TEF1 promoter identical to the endogenous TEF1 promoter of the fungus species, (2) a gene of interest, and (3) a terminator that is not the endogenous TEF1 terminator of the fungus species.

According to the invention, the terminator used in the expression cassette according to the invention is a terminator different from the endogenous TEF1 terminator of the fungus species (meaning the native TEF1 terminator of the fungus species). According to the invention, a TEF1 terminator from another fungus can therefore be used, provided that it is a TEF1 terminator different from the endogenous TEF1 terminator of the transformed fungus species (this is the case if the TEF1 terminator has a percentage identity of less than or equal to 75% compared to the sequence of the endogenous TEF1 terminator of the transformed fungus species). According to another particular embodiment, the terminator is different from an endogenous TEF1 terminator of a fungus species that is not the transformed fungus species. According to one particular embodiment, the terminator is a terminator different from a TEF1 terminator. In other words, it is not a terminator referred to as a TEF1 terminator in a species. For example, a CBH1 terminator can be used.

According to one particular embodiment, the integrative vector according to the invention (preferably a plasmid) comprises an expression cassette as mentioned above and a selection marker. According to one particular embodiment, the integrative vector according to the invention comprises an expression cassette as mentioned above, a non-fungal (for example bacterial) origin of replication, and a selection marker. The expression “selection marker” means a gene whose expression gives the cells containing it a characteristic which enables their selection. The selection marker is a marker compatible with the host. The use of a selection marker makes it possible to identify the fungi that have integrated a genetic modification, from those that have not integrated it. For example, this can be an antibiotic resistance gene, in particular the hph gene for resistance to the antibiotic hygromycin.

Fungi naturally possess a copy of the TEF1 gene, under the control of the TEF1 promoter. According to the invention, multicopy insertion is thus carried out in such a way that expression of the endogenous TEF1 gene of the transformed fungus species is maintained. In other words, the sequence encoding endogenous TEF1 and the endogenous TEF1 promoter are functional and show wild-type expression. According to one embodiment, in the method for inserting several copies of a gene of interest into the genome of a fungus, the insertion is carried out upstream of the endogenous TEF1 promoter in the genome of said fungus. Indeed, as indicated below, fungi naturally possess a copy of the TEF1 gene, under the control of the TEF1 promoter. According to this embodiment, multicopy insertion is therefore carried out upstream of the endogenous TEF1 promoter of the transformed fungus species. The insertion is thus made at 5′ of the endogenous TEF1 promoter. Preferably, in the method according to the invention, the insertion is thus carried out at between 750 and 1250 base pairs, preferably approximately 1000 base pairs, relative to the translation initiation site of the gene encoding TEF1. Preferably, in the method according to the invention, the insertion is thus carried out at between 750 and 1250 base pairs, preferably approximately 1000 base pairs, relative to the translation initiation site of the gene encoding the TEF1 protein of SEQ ID NO: 2 or a protein having a percentage identity of at least 80% with SEQ ID NO: 2.

The choice of the TEF1 promoter and the location of the multicopy insertion of the cassette as defined according to the invention, offer several advantages. (1) The TEF1 promoter has the advantage of being a strong promoter, thus allowing a large number of transcripts from the same carbon source. (2) Similarly to the TEF1 gene, the genes of interest controlled by the TEF1 promoter thus have a continuous, essentially constitutive expression, barely subject to repression systems. (3) The sequence of the TEF1 promoter also corresponds to the only flanking region necessary for targeting the TEF1 locus. According to one embodiment of the invention, the expression cassette according to the invention thus comprises a single flanking region for targeting the target locus of the insertion (region 5′ of the TEF1 promoter).

According to one embodiment of the invention, the endogenous TEF1 gene under the control of the endogenous TEF1 promoter are retained in the transformed genome of said fungus.

Among the fungi which can be used according to the invention, we can mention certain fungi of the phylum of Ascomycetes (Ascomycota), Basidiomycetes (Basidiomycota), and Zygomycetes (Zygomycota). In a preferred aspect, according to the invention, the fungus is a filamentous fungus. According to one embodiment, the filamentous fungus is selected among the classes Orbiliomycetes, Pezizomycetes, Dothideomycetes, Eurotiomycetes, Lecanoromycetes, Leotiomycetes, Sordariomycetes and Saccharomyces, preferably Sordariomycetes.

Advantageously, the fungus according to the invention belongs to the genus Trichoderma, and more particularly to the species Trichoderma reesei.

According to the invention, a “gene of interest” means any gene whose production is desired. Preferably, the gene of interest encodes a protein of interest. According to the invention, the proteins of interest are all the proteins which can be produced by a fungus naturally (such as enzymes) or else by genetic modification (for example after transformation using an appropriate vector). The protein of interest may be endogenous or exogenous. The protein of interest may be a native protein (as found in nature), a chimeric protein, a synthesized protein, or else a variant of a native protein, in particular exhibiting improved and/or modified biological properties. According to one embodiment, the sequence of the gene of interest may comprise appropriate additional elements such as, for example, a sequence encoding a secretion signal. The proteins of interest can be proteins useful for any type of industry (production of biofuel, food, cosmetics or pharmaceuticals, etc.

According to one embodiment, in the method according to the invention, the gene of interest encodes an enzyme, in particular a cellulolytic enzyme such as a cellulase or hemicellulase. Preferably, the enzymes are cellulases. According to the invention, the term “cellulases” more particularly means enzymes selected among endoglucanases, exoglucanases, and glucosidases, and more particularly β-glucosidase. The term “cellulase” more particularly refers to an enzyme adapted for the hydrolysis of cellulose and enabling the microorganisms (such as Trichoderma reesei) which produce them to use cellulose as a source of carbon, by hydrolyzing this polymer into simple sugars (glucose).

According to one embodiment, the method according to the invention comprises a step of selecting the transformed strain, after the transformation step.

The definitions and embodiments relating to the first aspect of the invention apply mutatis mutandis to the other aspects. For example, all definitions and preferences indicated in the first aspect of the invention above also apply to the second, third, fourth, fifth, sixth, and seventh aspect of the invention described below.

In a second aspect, the invention also relates to the use of an expression cassette to insert several copies of a gene of interest into the genome of a fungus, said expression cassette comprising:
a TEF1 promoter identical to the endogenous TEF1 promoter of the fungus species,
a gene of interest,
a terminator that is not the endogenous TEF1 terminator of the fungus species.

In a third aspect, the invention also relates to a method for producing a biomass, comprising the following steps:
(i) a step of transforming a fungus by using a vector comprising an expression cassette comprising: (1) a TEF1 promoter identical to the endogenous TEF1 promoter of the fungus species, (2) a gene of interest, and (3) a terminator that is not the endogenous TEF1 terminator of the fungus species,
(ii) a step of culturing the fungus in an appropriate culture medium.

According to one particular embodiment, said method for producing a biomass comprises a step of selecting a transformed fungus, between the transformation step and the culturing step.

According to one embodiment, the culture medium may comprise one or more substrates necessary for the growth of the fungus.

In a fourth aspect, the invention also relates to a method for producing a protein of interest, comprising the following steps:
(i) a step of transforming a fungus by using a vector comprising an expression cassette comprising: (1) a TEF1 promoter identical to the endogenous TEF1 promoter of the fungus species, (2) a gene of interest encoding said protein of interest, and (3) a terminator that is not the endogenous TEF1 terminator of the fungus species,
(ii) a step of culturing the fungus transformed in step (i), under conditions enabling production of the protein of interest.

According to one particular embodiment, said method for producing a protein of interest comprises a step of selecting a transformed fungus, between the transformation step and the culturing step.

The culturing step refers to culturing in an appropriate medium. This is a medium adapted for survival of the transformed fungus and/or for growth of the fungus and/or for production of the protein of interest, or even its secretion. It may be a medium adapted for culturing the fungus, supplemented as needed in order to become a medium suitable for the production and/or secretion of the protein of interest.

Advantageously, more particularly when the gene of interest encodes a cellulolytic enzyme, said method for producing a protein of interest also comprises a growth phase for growing a fungus strain transformed according to the invention, then a growth and production phase for growing and producing proteins of interest. Even more preferably, this growth phase is carried out in the presence of a growth substrate and this phase of growing and producing proteins of interest is carried out in the presence of an inducing substrate. The growth substrate and the inducing substrate are preferably carbonaceous substrates.

The carbonaceous growth substrate is thus preferably selected among lactose, glucose, xylose, the residues obtained after ethanolic fermentation of monomeric sugars from enzymatic hydrolysates of cellulosic biomass, and/or a crude extract of water-soluble pentoses coming from pretreatment of a cellulosic biomass.

The carbonaceous inducing substrate is thus preferably selected among lactose, cellobiose, sophorose, the residues obtained after ethanolic fermentation of monomeric sugars from enzymatic hydrolysates of cellulosic biomass, and/or a crude extract of water-soluble pentoses coming from pretreatment of a cellulosic biomass.

According to one embodiment, the method for producing a protein of interest may comprise a step of secreting said protein of interest. The secretion can be consecutive to or concomitant with the production of the protein of interest.

In a fifth aspect, the invention relates to a method for producing sugar from cellulosic or lignocellulosic substrates, comprising the following steps:
(i) a step of pretreating a cellulosic or lignocellulosic substrate in order to obtain a pretreated substrate,
(ii) a step of using a fungus transformed by using a vector comprising an expression cassette comprising: (1) a TEF1 promoter identical to the endogenous TEF1 promoter of the fungus species, (2) a gene of interest encoding a cellulolytic enzyme, and (3) a terminator that is not the endogenous TEF1 terminator of the fungus species,
(iii) a step of enzymatic hydrolysis of the pretreated substrate obtained in step i), in the presence of the cellulolytic enzymes obtained in step ii), in order to obtain a hydrolysate.

The sugars (or sugar juice) are thus obtained at the end of the hydrolysis step. Preferably they are C₅-C₆ sugars.

According to one embodiment, said method for producing sugars may also comprise one or more separation steps. This thus makes it possible to separate, in particular by distillation, the various products of interest diluted in water.

In a sixth aspect, the invention relates to a method for producing an alcohol from cellulosic or lignocellulosic substrates, comprising the following steps:
(i) a step of pretreating a cellulosic or lignocellulosic substrate in order to obtain a pretreated substrate,
(ii) a step of using a fungus transformed by using a vector comprising an expression cassette comprising: (1) a TEF1 promoter identical to the endogenous TEF1 promoter of the fungus species, (2) a gene of interest encoding a cellulolytic enzyme, and (3) a terminator that is not the endogenous TEF1 terminator of the fungus species,
(iii) a step of enzymatic hydrolysis of the pretreated substrate obtained in step i), in the presence of the cellulolytic enzymes obtained in step ii), in order to obtain a hydrolysate,
(iv) a step of alcoholic fermentation of the hydrolysate obtained,
(v) a step of separation, in particular by distillation.

The alcohol thus obtained is for example used as a biofuel. The alcohol thus obtained can also be used as a solvent or as an intermediate product in the chemical industry.

According to the invention, the term “biofuel” refers more specifically to a second-generation biofuel, meaning originating from non-food resources. According to the invention, the term “biofuel” can also be defined as being any product resulting from the transformation of biomass and which can be used for energy purposes. On the one hand and without limitation, we can cite as examples biogas, products which can be incorporated (possibly after subsequent transformation) into a fuel or which can be a fuel in their own right such as alcohols (ethanol, butanol, and/or isopropanol depending on the type of fermentation organism used), solvents (acetone), acids (butyric), lipids and their derivatives (short or long chain fatty acids, fatty acid esters), as well as hydrogen. Biofuel is thus an alcohol or a biogas.

Preferably, the alcohol is for example ethanol, butanol, isopropanol, 1,2-propanediol, 1,3-propanediol, 1,4-butanediol, and/or 2,3-butanediol. More preferably, it is ethanol.

According to a preferred embodiment, said steps iii) and iv) are carried out simultaneously. This is typically the case in “SSF” (Simultaneous Saccharification and Fermentation) production processes.

According to one particular embodiment, the step of pretreating a cellulosic or lignocellulosic substrate is a step of suspending in aqueous phase said cellulosic or lignocellulosic substrate.

According to one particular embodiment, the hydrolysate obtained in step iii) is a hydrolysate containing C₅-C₆ sugars, in particular glucose.

According to one particular embodiment, the step of alcoholic fermentation of the hydrolysate obtained is a step of fermentation, in the presence of a fermentative organism, of the glucose coming from the hydrolysate so as to produce a fermentation broth. A fermentative organism is for example a yeast.

According to one particular embodiment, the separation step is a separation of the biofuel and the fermentation broth, in particular by distillation.

According to an even more preferred embodiment, the cellulosic or lignocellulosic substrate to be hydrolyzed is suspended in aqueous phase at a content of 6 to 40% of dry matter, preferably to 30%. The pH is adjusted to be between 4 and 5.5, preferably between 4.8 and 5.2, and the temperature between 40° C. and 60° C., preferably between 45° C. and 50° C. The hydrolysis reaction is started by adding enzymes acting on the pretreated substrate. The amount of enzymes usually used is 10 to 30 mg of protein excreted per gram of pretreated substrate or less. The reaction generally lasts 15 to 48 hours. The reaction is monitored by assaying the released sugars, in particular glucose. The sugar solution is separated from the non-hydrolyzed solid fraction, essentially consisting of lignin, by filtration or centrifugation and then treated in a fermentation unit.

According to another even more preferred embodiment, when the hydrolysis and fermentation step are carried out together, the enzymes and the fermentative organism are added simultaneously and then incubated at a temperature between 30° C. and 35° C. to produce a fermentation broth. According to this embodiment, the cellulose present in the pretreated substrate is converted into glucose, and at the same time, in the same reactor, the fermentative organism (for example a yeast) converts the glucose into the final product according to a process of SSF (Simultaneous Saccharification and Fermentation) known to those skilled in the art. Depending on the metabolic and hydrolytic capacities of the fermentative organism, proper advancement of the operation may require the addition of a greater or lesser amount of exogenous cellulolytic mixture.

In a seventh aspect, the invention also relates to a strain as obtained by the method of the invention. The invention also relates to a fungus strain comprising, in its genome, several copies of a gene of interest under the control of a TEF1 promoter identical to the endogenous TEF1 promoter of the genetically modified fungus species.

The sequences of the invention are described in Table 1.

TABLE 1

| | |
|---|---|
| SEQ ID NO: 1 | AAAGCGGCGAATGACTCTTCGGGGTTGGCCAGAAACAACGACGACCACTGAGGGATCG |
| (TEF1 | GGACGATGTACATCTACTGCGATATGCCGGGCTCGAATGTGACTGCACACACCACACAC |
| promoter in | ACACACACACACGGACACATCTGAGCAGTACTATCAAAGCGGTGGGAGTGCATCTGCAC |
| Trichoderma | TGGGCACCCTTACTGTAGGTAGGAAGGATACCTAAGGTAGAGGGCTCGCTGCTGCAAGT |
| reesei) | TGATATGACGGCCACCAATCGGAAGGGTGTCGTATCTCACATCACCAGCGTCAGGACAC |
| | GCTTCACAAAATTCCCACAAGTTCCAGTTGAAATGACCCGTAGGAGAGCGCCTCGAGGG |
| | ACAGAATGTACAGTACTATACTACCTACCTTAGATGCCATCACCCCTTCGCAGGCCATGG |
| | AATTTCTGGCAAGTCGCACCCTTCAGCCATGCCTGTCCGCCATCATCATACCAGATCGTG |
| | TCGTGGCGGGCACTTGGGAAGGACGAGCTCCAACCGCCGCCGCTGATTGGCCTGATCC |
| | GCCACAGCCACTGGCGCTCGCGGCGAGGAAAAAAAAAAATCTTGCGGGTGTGCAGCAG |
| | CATTGAGTGGAAAGCGGGAGCCGCTGCACGACGCTGATTCGTCCGGGCTGCCTCGTTA |
| | GGGCTAGACCAGCAGGCGGGCGCCGCTTATTTTGCCCCTCCTCAGGCTTTTTTTCACAA |
| | GCCCCGTTTTGCTGCCCCGCGCGTGGCAGAAAAAAAAAATACAGCACGAGCCACTGGCA |
| | CTTGCACCCCTCCACCCCTCCGCCGACCACCTTTTAAATCTCCTCCTCCCACCATCTGCT |
| | TTCGAAATTTTCTCTCTTCTGCTCTTCGTCTCGCATACCCGGTTCAAGCATCCGATCTGCG |
| | AATTTGTATGTGCTGCCTCTCCCTCTGACCTTCTGGTCTGGTGATACCATCCTCCCTCAG |
| | TTTGGATCATCGCCTTATTCTTCTTCCCTCTTCTGCATCTGCTTCCTGCTCGTTTGAGGAA |
| | CATCGCCAGCTGACTCTGCTTGCCTCGCAGCGATCTAGTCAAGAACAACACAGCTCTCA |
| | CGCTACATCACACAAACCGTCAAA |
| SEQ ID NO: 2 | MAPQDYDDDDFVDQYDEDLGEEEDELSPEDKIAMQKGTEEVRKALGEEASKVTVAQIQEAL |
| (TEF1 protein | WHYYYDIDKSVAYLTRTFIAPPPKPAPKAAQKPAPKKAPEGKNGALSFFASGASFKPRIPPNF |
| in T reesei) | FDDMPWLNIPRDRETTFIEPPRLCGGLLGGTGSAPKVSKLQALAAMRKRKIEEKKAQENPDSL |
| | EGEVTKLALSDANQENTGTKSKIQRRNSSTDSSPPQEQPQKQGQNIPTASTDNSLGERVTLP |
| | TATESKTDHIIPQAPKPVMSRSHAPSAFAQALFESDSEADQRHRRDVFAMPYTQSSLFVASVF |
| | SEPSPDDIVLAARAKATPKTPKKSQKNEETSKDGALAGGVANLKVTDSPPPKSKGIDVIKEFE |
| | QSASKKHASFVVVGHVDAGKSTLMGRLLLELKLVQERTVDKYRRQAEKMGKTSFALAWVFD |
| | QRSEERDRGVTIDIATNHFETDSTNFTILDAPGHRDFVPNMIAGASQADFAVLVIDASTGAYEK |
| | GLKGQTKEHVLLLRSLGVQRIVVAVNKLEAVGWSQERFQEISEEISGFLTGLGFQEKSIKFIPIS |
| | GLNGDNIVKRSEDEACSWYTGPTLIEGLEASVPSTVRSLQKPFRMAISEIFRSQQGTTTLAGRI |
| | DSGTIQVGDALLVQPSGESAHVRSIMLDTETRDWAVAGQNVSIGLAGIDPVHVKVGDIVCHTK |
| | DPINVGDTFTMKAMAFAHLMPMPVDLHRGRLHAAGQIQSIAALLDKATGEVIKKKPKVVQPGS |
| | VARVLIKLDTKVPLEAGQRVVIRTGGETIAAGLLE |

### Sequences of the Invention

Other features, details, and advantages of the invention will become apparent upon reading the appended Figures and the examples which are intended solely to illustrate the invention without limiting it.

### BRIEF DESCRIPTION OF THE FIGURES

FIG. 1 represents the construction of the plasmid used in Example 1.

FIG. 2 shows the fluorescence intensity per unit size of germinating Trichoderma reesei spores. FIG. 2 shows the results for 8 transformants E1, E2, E3, E4, E5, E6, E8 and E9, and those for 3 non-transformed strains CL847-1, CL847-2 and CL847-3.

FIG. 3 shows the TEF promoter (TEF1) upstream of the TEF1 gene in the genome of a fungus.

FIG. 4 shows an illustration of integration into transformant E1. 7 copies of the gene of interest EGFP have been integrated. Indication of the following three items “(1) the TEF1 promoter, (2) the gene of interest EGFP, and (3) the terminator CBH1” illustrates that the cassette has been correctly integrated and that gene EGFP is functional.

FIG. 5 shows an illustration of integration into transformant E3. 10 copies of the gene of interest EGFP have been integrated. Indication of the following three items “(1) the TEF1 promoter, (2) the gene of interest EGFP, and (3) the terminator CBH1” illustrates the cases where the cassette has been correctly integrated and gene EGFP is functional.

### EXAMPLE

### Example 1: Construction of a Vector Containing an Expression Cassette according to the Invention

The following construction plasmid was obtained:
an origin of replication of Escherichia coli,
a selection marker comprising a fungal promoter “cpc” and bacterial promoter “IM7” followed by the Hygromycin resistance gene (hph) and the fungal terminator “TrpC”,
an expression cassette comprising: (1) a TEF1 promoter, (2) followed by gene EGFP expressing a green fluorescent protein, and (3) terminator CBH1.

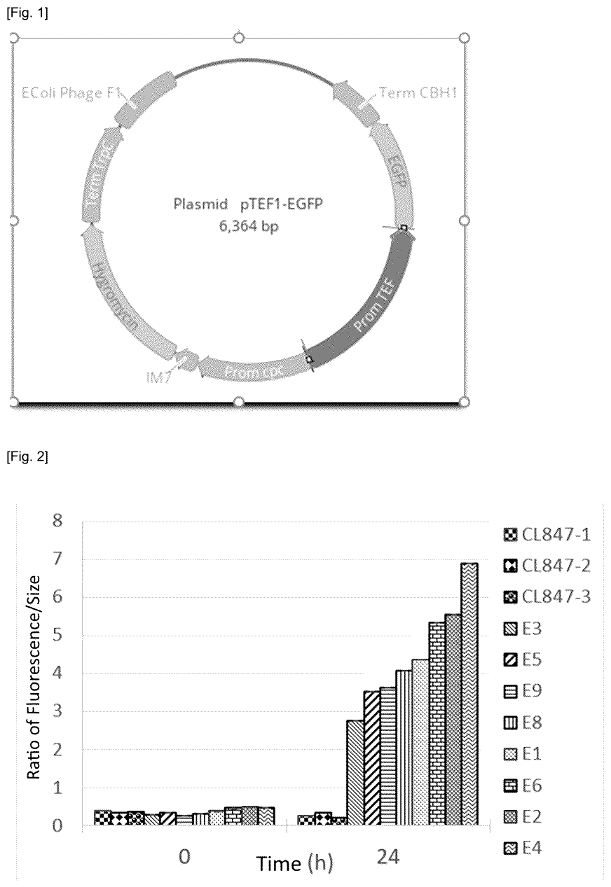

This vector can be obtained by any conventional method, well known to those skilled in the art. Construction of this plasmid is shown in FIG. 1. Moreover, a person skilled in the art can easily obtain a vector (plasmid type) comprising an expression cassette according to the invention and a selection marker making it possible to select the transformed fungi.

### Example 2: Transformation of a Fungus with the Expression Vector of Example 1

The plasmid of Example 1 was used to transform the strain of Trichoderma reesei referred to as CL847 (Durand H, Clanet M, Tiraby G. Genetic improvement of Trichoderma reesei for large scale cellulase production. Enzyme Microb Technol 1988, 10:341-346; Durand et al, 1984, Proc. Colloquium SFM “Génétique des microorganismes industriels”. Paris. H. HESLOT Ed, pp 39-50).

Transformants were selected by their ability to grow on a medium containing hygromycin.

The subcultures revealed different macroscopies between transformants on the dishes. After 3 subcultures, only those showing growth to confluence at the perimeter in 9 cm petri dishes, and displaying a dense, green and hydrophobic spore carpet were kept.

At the end of these 3 selection steps, eight transformants were kept and used for characterization by flow cytometry. The fluorescence study made it possible to reveal eight levels of fluorescence (FIG. 2).

The fluorescence intensity per unit size of germinating Trichoderma reesei spores is shown in FIG. 2. These measurements were made using a flow cytometer.

Genomic study of four of the eight transformants studied by flow cytometry made it possible to highlight the site of insertion, and the number of copies of the plasmid. In the four cases studied, all insertions were made upstream of the TEF1 promoter. The native and constitutive promoter of the TEF1 gene is preserved.

FIG. 3 illustrates the TEF1 gene under the control of the native and constitutive promoter of the TEF1 gene in the fungal genome.

FIG. 4 shows an illustration of the integration into transformant E1. 7 copies of gene of interest EGFP have been integrated

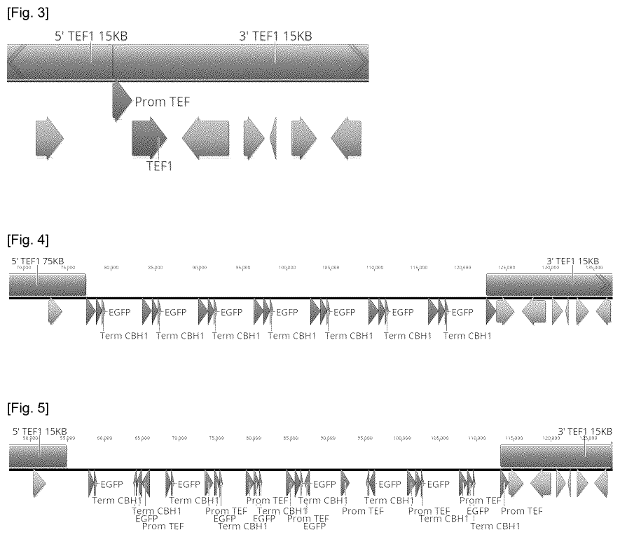

FIG. 5 shows an illustration of the integration into transformant E3. 10 copies of gene of interest EGFP have been integrated.

1. A method for inserting several copies of a gene of interest into the genome of a fungus, said method comprising a step of transforming said fungus by using a vector comprising an expression cassette, said cassette comprising:
a TEF1 promoter identical to the endogenous TEF1 promoter of the fungus species,
a gene of interest,
a terminator that is not the endogenous TEF1 terminator of the fungus species.

2. The method according to claim 1, wherein the insertion is carried out upstream of the endogenous TEF1 promoter in the genome of said fungus.

3. The method according to claim 1, wherein the endogenous TEF1 gene under the control of the endogenous TEF1 promoter are retained in the transformed genome of said fungus.

4. The method according to claim 1, wherein the TEF1 promoter is represented by:
SEQ ID NO: 1 or a sequence having a percentage identity of at least 60% with SEQ ID NO: 1, preferably at least 80%, or
the promoter of a gene encoding a protein of SEQ ID NO: 2 or a protein having a percentage identity of at least 80% with SEQ ID NO: 2.

5. The method according to claim 1, wherein the fungus is selected among the classes Orbiliomycetes, Pezizomycetes, Dothideomycetes, Eurotiomycetes, Lecanoromycetes, Leotiomycetes, Sordariomycetes and Saccharomyces.

6. The method according to claim 1, wherein the fungus belongs to the genus Trichoderma.

7. The method according to claim 1, wherein the gene of interest encodes an enzyme.

8. A method for inserting several copies of a gene of interest into the genome of a fungus, with an expression cassette comprising:
a TEF1 promoter identical to the endogenous TEF1 promoter of the fungus species,
a gene of interest,
a terminator that is not the endogenous TEF1 terminator of the fungus species.

9. A method for producing a protein of interest, comprising the following steps:
(i) a step of transforming a fungus by using a vector comprising an expression cassette comprising: (1) a TEF1 promoter identical to the endogenous TEF1 promoter of the fungus species, (2) a gene of interest encoding said protein of interest, and (3) a terminator that is not the endogenous TEF1 terminator of the fungus species,
(ii) a step of culturing the fungus transformed in step (i), under conditions enabling production of the protein of interest.

10. A method for producing a biomass, comprising the following steps:
(i) a step of transforming a fungus by using a vector comprising an expression cassette comprising: (1) a TEF1 promoter identical to the endogenous TEF1 promoter of the fungus species, (2) a gene of interest, and (3) a terminator that is not the endogenous TEF1 terminator of the fungus species,
(ii) a step of culturing the fungus in an appropriate culture medium.

11. A method for producing sugar from cellulosic or lignocellulosic substrates, comprising the following steps:
(i) a step of pretreating a cellulosic or lignocellulosic substrate in order to obtain a pretreated substrate,
(ii) a step of using a fungus transformed by using a vector comprising an expression cassette comprising: (1) a TEF1 promoter identical to the endogenous TEF1 promoter of the fungus species, (2) a gene of interest encoding a cellulolytic enzyme, and (3) a terminator that is not the endogenous TEF1 terminator of the fungus species,
(iii) a step of enzymatic hydrolysis of the pretreated substrate obtained in step i), in the presence of the cellulolytic enzymes obtained in step ii), in order to obtain a hydrolysate.

12. A method for producing an alcohol from cellulosic or lignocellulosic substrates, comprising the following steps:
(i) a step of pretreating a cellulosic or lignocellulosic substrate in order to obtain a pretreated substrate,
(ii) a step of using a fungus transformed by using a vector comprising an expression cassette comprising: (1) a TEF1 promoter identical to the endogenous TEF1 promoter of the fungus species, (2) a gene of interest encoding a cellulolytic enzyme, and (3) a terminator that is not the endogenous TEF1 terminator of the fungus species,
(iii) a step of enzymatic hydrolysis of the pretreated substrate obtained in step i), in the presence of the cellulolytic enzymes obtained in step ii), in order to obtain a hydrolysate,
(iv) a step of alcoholic fermentation of the hydrolysate obtained,
(v) a step of separation, in particular by distillation,
steps (iii) or (iv) possibly being carried out simultaneously.

13. A fungus strain comprising, in its genome, several copies of a gene of interest under the control of a TEF1 promoter identical to the endogenous TEF1 promoter of the genetically modified fungus species.

14. The method according to claim 2, wherein the insertion is carried out at between 750 and 1250 bp relative to the translation initiation site of the gene encoding TEF1 protein.

15. The method according to claim 14, wherein the TEF1 protein is represented by SEQ ID NO: 2 or by a protein having at a percentage identity of at least 80% with SEQ ID NO: 2.

16. The method according to claim 1, wherein the fungus belongs to the specifies Trichoderma reesei.

17. The method according to claim 7, wherein said enzyme is a cellulolytic enzyme such as a cellulase or hemicellulose.
